(19) United States
(12) Patent Application Publication
(10) Pub. No.: US 2012/0083007 A1
(43) Pub. Date: Apr. 5, 2012

(54) Basophil Activation Based Allergy Diagnostic Test

(76) Inventors: Kari C. Nadeau, Stanford, CA (US); Amit Kumar Singh, Camden Town (GB)

(21) Appl. No.: 13/246,525

(22) Filed: Sep. 27, 2011

Related U.S. Application Data
(60) Provisional application No. 61/413,826, filed on Nov. 15, 2010.
(60) Provisional application No. 61/389,128, filed on Oct. 1, 2010.

(51) Int. Cl. G01N 33/53

(52) U.S. Cl. 435/7.24

(57) ABSTRACT

Methods are provided for determining a subject's susceptibility to an allergic reaction upon exposure to an offending allergen. Methods are also provided for determining and monitoring a subject's responsiveness to ongoing allergy treatment.

### BACKGROUND

The analysis of subpopulations of white blood cells (leukocytes) in blood or bone marrow is of particular interest for the evaluation of immune system disorders and immune system-related diseases, especially of allergic diseases. Granulocytes are a subpopulation of leukocytes and encompass neutrophils, eosinophils as well as basophils, which all differ in their staining characteristics and abundance in blood.

Neutrophils are the most abundant white blood cells in humans and account for approximately 70% of white blood cells, while basophils and eosinophils are much rarer, accounting for less than 1 percent and 1-6% of white blood cells, respectively.

Both basophils and eosinophils play important roles in Immunoglobulin E-mediated (IgE-mediated) immune responses including food allergies, severe asthma and responsiveness to environmental allergens. The basophil and/or eosinophil count often increases prior to the onset of symptoms and the activation state of basophils and eosinophils may correlate with the symptoms.

Allergies of diverse causes are on the rise particularly in developed countries as a consequence of heightened susceptibility towards aeroallergens, (heavy) metals, metal alloys, food allergens, xenobiotics, microbial allergens and more. The identification and quantitation of IgE antibodies is central in common tests to diagnose allergy; however, not all allergies are IgE-mediated.

Food allergy is a major public health problem that affects as many as 3-4% of adults and 6% of children in the United States. The incidence of allergic diseases and food allergies has increased 10-fold in developed countries in the last two decades. Currently used, first-line methods for identifying offending antigens are based on in-vivo as well as in-vitro allergen tests.

The in-vivo allergen testing is usually carried out as a skin test, which is typically uncomfortable for the patient, in particular for the pediatric patient, and often inconclusive. In-vitro allergen tests include radio-allergo sorbent test (RAST), immunoCAP and histamine liberation tests. These assays are blood-based, typically require several milliliters of blood and several days until the test results are available; in addition, none of these have proven reliable in terms of specificity and sensitivity.

If these first-line assays remain inconclusive, an in-vivo food challenge test is carried out in a double-blind, placebo-controlled fashion to determine the offending allergen. This test is not only difficult to administer, it is also very time-consuming and, most importantly, potentially highly dangerous since it can result in anaphylactic shock and even death, if treatment is not initiated quickly.

Taken together, the identification of an offending allergen is not always possible and not all allergies are IgE-mediated, rendering many common in-vitro and in-vivo tests inadequate for the specific and reliable determination of the causes of allergic reactions. Consequently, there is a great, currently unmet need for specific, sensitive, safe and rapid methods to determine a subject's susceptibility to possibly offending allergens of diverse origin.

### SUMMARY

The present invention addresses the currently unmet need for specific, sensitive, safe and rapid methods to determine a subject's susceptibility to possibly offending allergens of diverse origin.

In some embodiments, a method is provided for ex vivo determination of a subject's susceptibility to an allergic reaction upon exposure to an allergen of interest. The subject may have a suspected allergy, or may have no known allergy to the allergen or known predisposition. The method is based on the monitoring and detection of a cell surface protein on the surface of viable basophils, eosinophils and/or other granulocytes from the subject, following stimulation by the allergen of interest.

Blood, e.g. whole blood, is collected from the subject, and incubated with the allergen. Blood cells are then analyzed for expression of cell surface markers indicative of an allergic response, for example CD203c, CD63, etc. In some embodiments the markers are analyzed by contacting with an antibody specific for the cell surface marker, washed of unbound antibody, and checked by flow cytometry, microscopy, MACS, etc. to determine the presence of bound antibody, indicative of the presence of the cognate marker. The methods find use in determining human allergies to a variety of allergens, which include without limitation food, environment, microbial, nano-particles, metals, drug molecules, etc. The methods of the invention can be used in the diagnosis, and monitoring of allergies or related diseases in humans.

In some embodiments of the invention, the methods are used in the diagnosis of allergies in an individual. In other embodiments the methods are utilized in the screening of candidate agents for activity in altering an allergic response. In other embodiments the methods are utilized in the monitoring of effectiveness of current therapeutic modalities, e.g. with respect to the responsiveness of an individual being treated. The methods of the invention also find use in research methods, e.g. to test mechanisms of specific pathways in basophils.

Advantages of the methods are the simplicity, ease of use and small volume. Only a small sample volume is required to perform the basophil activation. Multiple allergens and patients can be tested at the same time by using 96 well plate or any multi well plates or tubes. Two or three colors are used in the flow cytometry assay, which makes the assay economically viable and adaptable to clinical lab use. The method is fast and requires less than one hour to complete, and it is reliable and efficient.

In some embodiments, basophils are identified by a single antibody in a flow cytometry assay. In other embodiments, multiple antibodies are used, so as to effectively “gate” the analysis on basophils, for example where the activated cell sample is contacted with markers that distinguish between basophils and other blood cells. Scatter profiles also find use in gating.

Diseases and disorders where this method will be useful are food allergy, airborne allergy, drug-induced allergy mediated through the ingestion, inhalation, injection or skin exposure to a xenobiotic, anaphylaxis, asthma and other immune disorders caused by microbes or contact with (heavy) metals or metal alloys.

Particular embodiments of the invention use very small volumes of blood (100 μl or less per assay) and so are also suitable for studies in all type of subjects (e.g. infants, children, healthy and sick individuals).

All publications and patent applications mentioned in this specification are herein incorporated by reference to the same extent as if each individual publication or patent application was specifically and individually indicated to be incorporated by reference.

### DESCRIPTION OF THE DRAWINGS

FIG. 1: Induction of CD203c after allergen stimulation: Top panel showing CCR3^{+high}/SSC^{low} cells to recognize basophils. Bottom panel shows expression of CD203c before and after stimulation with offending and non-offending allergens after 20 minutes.

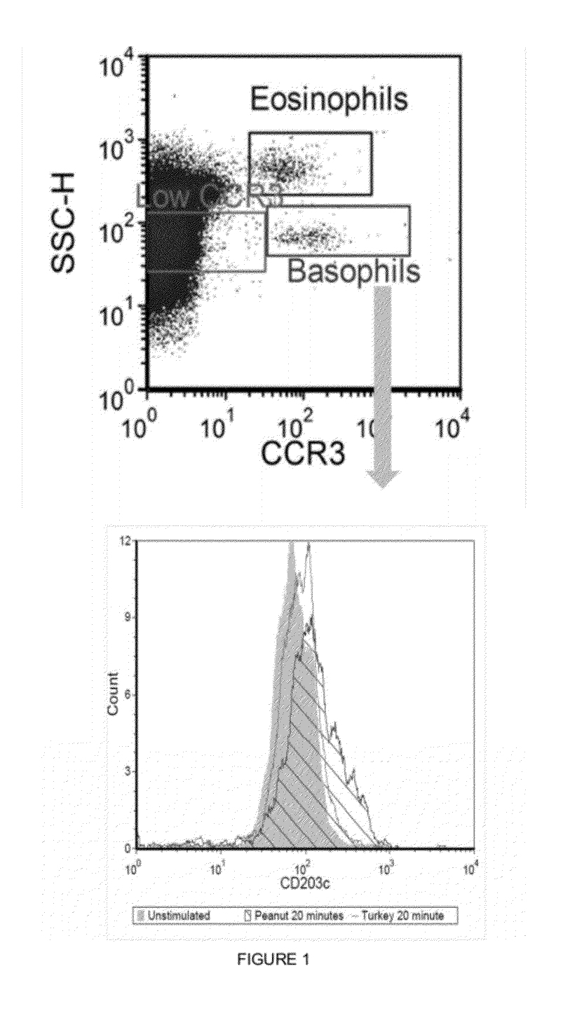

FIG. 2A-B. Levels of CD203c (A) and CD63 (B) surface expression on basophils measured 20 min after ex vivo peanut stimulation were increased in PA subjects with more severe reactions to the DBPCFC performed at screening (n=25 subjects total are shown with clinical history of peanut allergies who underwent a DBPCFC, 13 had grade 1 reactions, 5 with grade 2, 4 with grade 3, and 3 had a grade 4 reaction); In addition, 5 healthy controls with no clinical history of peanut allergies are shown and predictably had a grade 0 when performing the DBPCFC.

FIG. 3A-D. Basophil whole blood test (gated from whole blood by CCR3 and CD123) were analyzed by flow cytometry using CD203c as an activation marker 20 min after TG (timothy grass) or DM (dust mite) stimulation ex vivo. Data are from 20 SLIT subjects. P<0.001 for (A). Red box=non-tolerant subjects (n=7) after stopping SLIT for 6 mo. All active subjects, including the 10 taken off therapy from month 12 to month 18 were on dual SLIT therapy from 18 mo to 24 mo to see if basophil reactivity could be reversed.

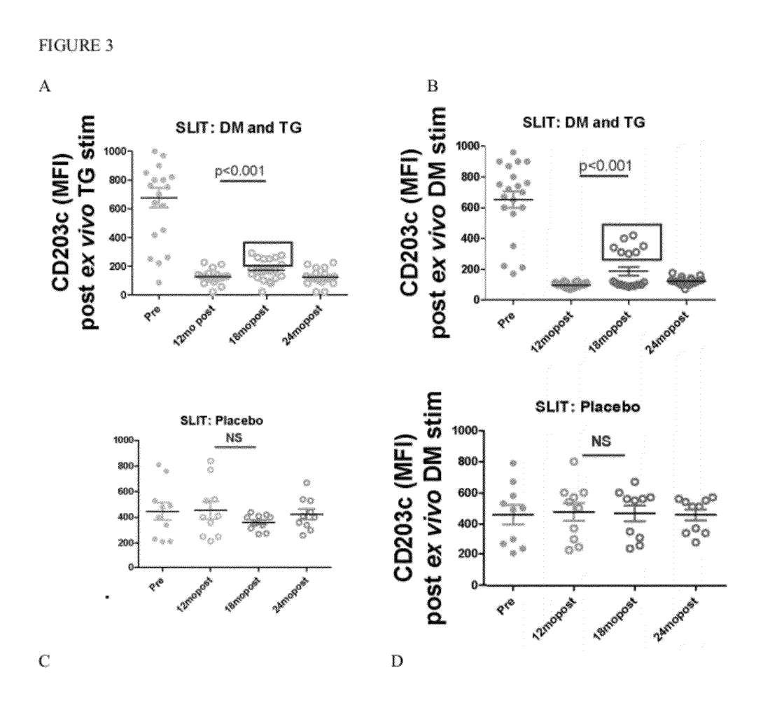

### DEFINITIONS

The practice of the present invention may employ conventional techniques of chemistry, molecular biology, recombinant DNA, microbiology, cell biology, immunology and biochemistry, which are within the capabilities of a person of ordinary skill in the art. Such techniques are fully explained in the literature. For definitions, terms of art and standard methods known in the art, see, for example, Sambrook and Russell ‘Molecular Cloning: A Laboratory Manual’, Cold Spring Harbor Laboratory Press (2001); ‘Current Protocols in Molecular Biology’, John Wiley & Sons (2007); William Paul ‘Fundamental Immunology’, Lippincott Williams & Wilkins (1999); M. J. Gait ‘Oligonucleotide Synthesis: A Practical Approach’, Oxford University Press (1984); R. Ian Freshney “Culture of Animal Cells: A Manual of Basic Technique”, Wiley-Liss (2000); ‘Current Protocols in Microbiology’, John Wiley & Sons (2007); ‘Current Protocols in Cell Biology’, John Wiley & Sons (2007); Wilson & Walker ‘Principles and Techniques of Practical Biochemistry’, Cambridge University Press (2000); Roe, Crabtree, & Kahn ‘DNA Isolation and Sequencing: Essential Techniques’, John Wiley & Sons (1996); D. Lilley & Dahlberg ‘Methods of Enzymology: DNA Structure Part A: Synthesis and Physical Analysis of DNA Methods in Enzymology’, Academic Press (1992); Harlow & Lane ‘Using Antibodies: A Laboratory Manual: Portable Protocol No. I’, Cold Spring Harbor Laboratory Press (1999); Harlow & Lane ‘Antibodies: A Laboratory Manual’, Cold Spring Harbor Laboratory Press (1988); Roskams & Rodgers ‘Lab Ref: A Handbook of Recipes, Reagents, and Other Reference Tools for Use at the Bench’, Cold Spring Harbor Laboratory Press (2002). Each of these general texts is herein incorporated by reference.

Unless defined otherwise, all technical and scientific terms used herein have the same meaning as commonly understood by a person of ordinary skill in the art to which this invention belongs. The following definitions are intended to also include their various grammatical forms, where applicable.

The term “subject”, “mammalian subject”, “individual” or “patient” are used interchangeably herein to refer to a member of a species of mammalian origin, including but not limited to a human, mouse, rat, cat, goat, sheep, horse, hamster, ferret, pig, dog, guinea pig, rabbit or primate, adult or not yet adult.

The terms “allergic response” and “allergy” are used interchangeably herein to describe an abnormal reaction of the body to a previously encountered allergen introduced by inhalation, ingestion or skin contact. The use of these terms also includes clinically adverse reactions to environmental allergens which reflect the expression of acquired immunologic responsiveness involving allergen-specific antibodies and/or T cells. These terms also include adverse immunologic responses that are associated with the production of allergen-specific IgE.

The term “allergen”, as used herein, refers to any substance that induces an allergy in a susceptible subject. The use of the term “allergen” includes any antigens that elicit a specific IgE response. Allergens may have little or no intrinsic toxicity by themselves, but cause a pathological condition due to their ability to elicit an IgE-associated immune response, and, upon subsequent exposure, due to their ability to elicit IgE- and/or T cell-dependent hypersensitivity reactions. Common allergens include but are not limited to pollen, grasses, dust, as well as foods, including, but not limited to, nuts, milk, eggs, shell fish, and venoms, and various drugs. Allergens include, without limitation, nanoparticles, metal or metal alloys, drug or medicine related antigens; various biological matters, e.g. proteins, which may be related to animals such as insects or arachnids. Other allergens may be related to humidifiers and air conditioners.

The term “allergic diseases”, as used herein, refers to a group of clinically manifested disorders in which immune responses, typically directed against otherwise innocuous environmental allergens, are thought to have a pathogenetic role. Allergic diseases include, but are not limited to, hay fever, allergic asthma, allergic contact dermatitis, and clinical disorders in which IgE-associated immune responses are thought to play a role.

The term “activation”, as used herein, refers to a physiological condition upon exposure to a substance, allergen, drug, protein, chemical, or other stimulus, or upon removal of a substance, allergen, drug, protein, chemical or other stimulus.

Positive control antigen, as used herein refers to anti-IgE, anti-IgG, anti-IgD antibodies, fMLP, cytokines, IL-3, IL-18, IL-33, histamine, anti-Fc receptor antibodies, PMA/Ionomycin, PMA/ca1, proteases enzymes, papain, TLR receptor/ligand or antibodies against TLR or agonists, complement factors, antigens from helmiths, ROS pathway markers, or other intracellular or extracellular markers that are involved in the basophil activation or degranulation.

The term “activation marker”, as used herein, refers to cell surface markers indicative of basophil activation, for example one or both of CD203c and CD63; CD13, CD107a, CD164, CD80, CD86, CD40L, HLA-DR, CD123, CRTH2 and other extracellular markers on basophils, or intracellular markers such as Ph-CREB, Ph-STATS, Ph-S6rp, Ph-eIF4E, CREB or mTOR pathway proteins, or other phosphorylation related markers, or other proteins or small molecules related to the activation of basophils. CD203c and CD63 are of particular interest. The physiological condition may be the result of an exposure to a substance, allergen, drug, protein, chemical, or other stimulus, or maybe the result of removal of a substance, allergen, drug, protein, chemical or other stimulus.

The term “cell surface marker”, as used herein, refers to an antigenic determinant or epitope found on the surface of a specific type of cell. Cell surface markers can facilitate the characterization of a cell type, its identification, and eventually its isolation. Cell sorting techniques are based on cellular biomarkers where one or more cell surface markers are used for either positive or negative selection, i.e., for inclusion or exclusion, from a cell population.

The term “cytometry”, as used herein, refers to a process in which physical and/or chemical characteristics of single cells, or by extension, of other biological or nonbiological particles in roughly the same size or stage, are measured. In flow cytometry, the measurements are made as the cells or particles pass through the measuring apparatus (flow cytometer) in a fluid stream. A cell sorter, or flow sorter, is a flow cytometer that uses electrical and/or mechanical means to divert and collect cells (or other small particles) with measured characteristics that fall within a user-selected range of values.

A marker that distinguishes basophils from other blood cells refers to a detectable physical parameter, particularly a parameter that can be monitored by flow cytometry, that allows basophils to be “gated” or separately analyzed from other blood cells. Markers of particular interest include size (detectable by forward scatter), granularity (detectable by side scatter) and cell surface markers, which can be detected by antibody staining for the marker of interest. In some embodiments of the invention, a cell sample is stained with one or more antibodies that distinguish between basophils and T cells, such as CCR3, CD3, CD4, CD8, HLA-DR, CD123 and the like, which may be combined with side scatter for gating. For example, the data points of a flow cytometry analysis may be limited to those cells that stain for CCR3, and are not high or low in granularity.

A gate in cytometry is a set of value limits (boundaries) that serve to isolate a specific group of cytometric events from a large set. Gates can be defined by discrimination analysis, or can simply be drawn around a given set of data points on a print-out and then converted to a computer-useful form. Gates can be implemented with a physical blinder. Gates may be used either to selectively gather data or to segregate data for analysis. Gates are divided mathematically into inclusive gates and exclusive gates. Inclusive gates select data that falls within the limits set, while exclusive gates select data that falls outside the limits. A live gate is a term used for a process that prevents the acquisition by the computer of non-selected data from the flow cytometer. (see, for example, Osborne, G. W. (2000) “Regions and Gates” Flow Cytometry Software Workshop: 2000, page 3).

The term “active” or “activated”, as used herein, refers to having a biological or physiological effect that differs from the native biological, physiological, or wildtype, state.

The term “nonactivated”, as used herein, refers to a native biological, physiological, or wildtype, state.

The term “activatable”, as used herein, refers to having potential to become biologically or physiologically active.

The term “normal”, as used herein, refers to a standard, model, median or average of a large group. “Abnormal”, as used herein, refers to a deviation of the standard, model, median or average of a large group.

The term “antigen”, as used herein, refers to any substance that can stimulate the production of antibodies and can combine specifically with them. The term “antigenic determinant” or “epitope”, as used herein, refers to an antigenic site on a molecule.

The term “biological sample”, as used herein, refers to a sample consisting of or containing blood, serum, plasma, lymph fluid, amniotic fluid, saliva, cerebro-spinal fluid, lacrimal fluid, mucus, urine, sputum, or sweat.

The term “drop”, as used herein, refers to a small quantity of liquid or liquid globule that is produced, or falls, in a more or less spherical mass.

The term “contacting”, as used herein, refers to a state of touching or immediate or local proximity.

The term “disease” or “disorder”, as used herein, refers to an impairment of health or a condition of abnormal functioning.

The term “drug”, as used herein, refers to a therapeutic agent or any substance, other than food, used in prevention, diagnosis, alleviation, treatment or cure of disease.

The term “differential label”, as used herein, generally refers to a stain, dye, marker, or antibody used to characterize or contrast structures, components or proteins of a single cell or organism.

The term “labeling”, as used herein, refers to a process of distinguishing a compound, structure, protein, peptide, antibody, cell or cell component by introducing a traceable constituent. Common traceable constituents include, but are not limited to, a fluorescent antibody, a fluorophore, a dye or a fluorescent dye, a stain or a fluorescent stain, a marker, a fluorescent marker, a chemical stain, a differential stain, a differential label, and a radioisotope.

The term “stain”, as used herein, refers to a composition of one or more dyes or pigments used to make differentiable a structure, a material, a cell, a cell component, a membrane, a granule, a nucleus, a cell surface receptor, a peptide, a microorganism, a nucleic acid, a protein or a tissue.

The term “susceptible”, as used herein, refers to a member of a population at risk. The term is inclusive of a subject having a medical history of a previous allergic reaction to at least one allergen and at risk of mounting an allergic reaction to a different antigen.

The term “anaphylactic shock”, as used herein, refers to a sudden, severe allergic reaction typically characterized by a sharp drop in blood pressure, urticaria, and breathing difficulties that are caused by exposure to a foreign substance after a preliminary or sensitizing exposure.

The term “expression”, as used herein, refers to the action of a gene in the production or a protein or phenotype. “Level of expression” refers to the degree to which a particular gene produces its effect(s) in an organism.

The term “dye”, as used herein, (also referred to as “fluorochrome” or “fluorophore”) refers to a component of a molecule which causes the molecule to be fluorescent. The component is a functional group in the molecule that absorbs energy of a specific wavelength and re-emits energy at a different, but equally specific wavelength. The amount and wavelength of the emitted energy depend on both the dye and the chemical environment of the dye.

The term “fluorescent-activated cell sorting” (also referred to as “FACS”), as used herein, refers to a method for sorting a heterogeneous mixture of biological cells into one or more containers, one cell at a time, based upon the specific light scattering and fluorescent characteristics of each cell.

The term “isolated”, as used herein, refers to placing, setting apart, or obtaining a protein, molecule, substance, nucleic acid, peptide, cell or particle, in a form essentially free from contaminants or other materials with which it is commonly associated.

The term “stimulation”, as used herein, describes the addition of a defined amount of test allergens/antigens to a blood sample from patients with suspected allergies and subsequent incubation at controlled temperature.

The term “venipuncture”, as used herein, refers to the process of obtaining intravenous access for the purpose of intravenous therapy or obtaining a sample of venous blood.

The term “whole blood”, as used herein, refers to generally unprocessed or unmodified collected blood containing all of its components, such as red blood cells, white blood cells, platelets and plasma. The term “whole blood” is inclusive of any anticoagulant that may be combined with the blood upon collection.

Enhancing agent, as used herein, refers to growth factors and interleukins, for example, IL-3, at a dose sufficient to increase activation markers, as described above, after 10 minutes or more of incubation of enhancing agent and test allergen with whole blood.

### DETAILED DESCRIPTION OF THE EMBODIMENTS

Methods of the invention provide an ex vivo, whole blood-based method of determining a subject's susceptibility to an allergic reaction upon exposure to an allergen of interest. The method is based on the monitoring and detection of cell surface expression of markers on one or more of live basophils, eosinophils and/or neutrophils from the subject following stimulation by the allergen.

Particular embodiments of the invention determine granulocyte response to the binding of allergen to allergen-specific IgE or other relevant Ig in whole blood, without prior isolation of granulocytes. There is no need to pre-isolate the granulocytes, since their activation is detected by flow cytometric assays that enable functional isolation and detection of specific signaling in granulocytes in small amounts of appropriately stimulated whole blood.

According to one aspect, the present invention provides a method for determining a subject's response to an allergen challenge in a whole blood sample obtained from a subject suspected of an allergy. The method comprises the steps of collecting a blood sample from the subject, contacting the blood sample with an allergen, assessing the level of expression of a cell surface marker that is characteristic to the activation of basophilic, neutrophilic or eosinophilic granulocytes and correlating the expression of the marker with the subject's response to the allergen challenge. The expression may be compared, for example, to a control sample in which allergen is not present. In some embodiments the response of all granulocytes is monitored. In other embodiments, the analysis is gated on basophils.

The blood sample that provides a source of basophils may be obtained by any convenient method. A benefit of the invention is the ability to work with small volumes, usually from about 50 μl to about 500 μl is used, which may be an aliquot of a larger sample volume, and may be from about 100 μl to about 250 μl. For the analysis, whole blood may be used, or a basophil enriched blood sample. A anti-clotting agent is typically added to the sample, e.g. EDTA, heparin, and the like, as are known and used in the art. Optionally platelets and red blood cells are centrifuged out from the sample. Blood is preferably used within about 72 hours from collection, and will generally be frozen, kept on ice, etc.

The candidate allergen is added to the blood sample, typically at a range of concentrations. Preferred allergen extracts are standardized, and are commercially available (for example from Greeg Pharmaceuticals, etc.). A positive control is usually performed as well, for example using polyclonal IgE at 1 μg/ml. The cells are incubated with the allergen for a period of time sufficient to provide basophil activation, usually at least about 10 minutes, at least about 15 minutes, and not more than about 1 hour, usually not more than about 45 minutes, or not more than about 30 minutes. The activation process can be stopped by the addition of, for example azide or a similar agent.

The sample is then contacting with binding agents that selectively bind to (a) a marker for basophil activation; and (b) a marker that distinguishes basophils from other blood cells. Antibodies are a preferred reagent, and may be directly or indirectly labeled. Included as marker for basophil activation are one or more of CD203c, CD63, CD13, CD107a, CD164, CD80, CD86, CD40L, HLA-DR, CD123, CRTH2, Ph-CREB, Ph-STATS, Ph-S6rp, Ph-eIF4E, CREB , mTOR pathway proteins, and phosphorylation related markers. In preferred embodiments the marker for basophil activation is one or both of CD203c and CD63.

Markers that distinguish basophils from other blood cells include CCR3, HLA-DR, CD123, and the like. In some embodiments the basophils are gated as CCR3^{high}/SSC^{low}, in other embodiments the basophils are gated as HLA-DR^{negative}/CD123^{positive}.

The cells are analyzed by any convenient method, which includes flow cytometry. An allergen activation is usually indicated as present when the increase in level of a basophil activation marker is at least 10-fold the level in the absence of allergen, at least 20-fold, at least 30-fold or more.

### Immune System Disorders and Immune-System Related Diseases Allergy

### Allergy

Allergy is a disorder of the immune system and is characterized by the occurrence of allergic reactions to normally harmless environmental substances known as allergens; these reactions are acquired, predictable, and rapid. Allergies are caused by allergens, which may be present in a wide variety of sources, including but not limited to pollens or other plant components, dust, moulds or fungi, foods, additives, latex, transfusion reactions, animal or bird danders, insect venoms, radiocontrast medium, medications or chemicals. Common allergic reactions include eczema, hives, hay fever, asthma, food allergies and reactions to venoms. Mild allergies like hay fever are highly prevalent in the human population and cause symptoms such as allergic conjunctivitis, itchiness, and runny nose. In some people, severe allergies to environmental or dietary allergens or to medication may result in life-threatening anaphylactic reactions and potentially death, if left untreated. Allergic reactions can occur in three distinct patterns: a) an early phase reaction or acute response, b) late phase reactions and c) potentially chronic allergic inflammation. The early phase of the allergic reaction typically occurs within minutes, or even seconds, following a first allergen exposure and is also commonly referred to as the immediate allergic reaction. In the early stages of allergy, a hypersensitivity reaction against an allergen, encountered for the first time, cause a response in Th2 cells, which are a subset of T cells that produce the cytokine interleukin-4 (IL-4). The Th2 cells interact with B cells (lymphocytes that produce antibodies against antigens) and, coupled with the effects of IL-4, stimulate the B cells to begin production and secretion of Immunoglobulin E (IgE).

IgE plays an important role in allergies and allergic reactions. Upon introduction of an allergen, B cells of the respective subject produce large amounts of IgE. The IgE elicits an immune response by binding onto receptors found on basophils and mast cells. When activated, these cells release chemical mediators such as histamine and cytokines that cause the characteristic symptoms of allergy.

Immunoglobulin G (IgG) is an abundant immunoglobulin in humans that protects the individual from pathogens such as viruses, bacteria, and fungi. Upon introduction of a pathogen, IgG binds to the pathogen, allowing for various defense mechanisms such complement activation, opsonization for phagocytosis, and neutralization of pathogen toxins. IgG also plays a role in food allergies. In contrast to IgE-mediated allergic reactions to food, whose symptoms appear rapidly, IgG-mediated allergic reactions to food have a delayed onset. IgG can be divided into various subclasses based on their effector functions.

The secreted IgE circulates in the blood and binds to the high affinity IgE receptor (FcεRI) on the surface of mast cells and basophils, both of which are involved in the acute inflammatory response. At this state, the IgE-coated cells are sensitized to the allergen. Mast cells are very similar to basophils; however, current evidence suggests that they are generated by different precursor cells in the bone marrow.

If later exposure to the same allergen occurs, the allergen can bind to the IgE molecules held on the surface of the mast cells or basophils. Cross-linking of the IgE and high affinity IgE receptors occurs when more than one IgE-receptor complex interacts with the same allergenic molecule, and activates the sensitized cell. Subsequently, these activated mast cells and basophils undergo the process of degranulation during which they release histamine and other inflammatory chemical mediators, such as cytokines, interleukins and prostaglandins, from their granules into the surrounding tissue causing several systemic effects, such as vasodilation, mucous secretion, nerve stimulation, and smooth muscle contraction. This may result in rhinorrhea, itchiness, dyspnea, or anaphylaxis. Depending on the individual subject, allergen and mode of introduction of the allergen, the symptoms may be system-wide or localized to a particular body system.

After the chemical mediators of the acute response subside, late phase responses may occur. Tissue may become red and swollen due, initiated by the release of cytokines from mast cells and basophils, to the migration of other white blood cells such as neutrophils, lymphocytes, eosinophils and macrophages to the initial site. The reaction can occur between 2 to 24 hours following repeated contact with an offending allergen. Chronic allergic inflammations can persist for days to years. Once a subject is sensitized, i.e. has been exposed repeatedly to an offending allergen, a range of tissue responses might ensue, depending on such factors as the route, frequency and extent of allergen exposure, and on whether the allergen exposure represents a single transient occasion, results in the persistence of the allergen, or occurs seasonally, e.g. as in the case of hay fever, or in some other repetitive fashion. Tissue responses may also be affected by the genetic background of the subject and by diverse nongenetic factors, such as a concurrent infection, which might modify the subject's response to an allergen.

### Allergy Treatments

Treatments for allergies include allergen avoidance, local or internal use of anti-histamines, local or internal use of corticosteroids, immunotherapy to (gradually) desensitize the response to allergen, and targeted pharmacological intervention.

### Monitoring of Allergy Treatment Success

Consistent allergen avoidance would be ideal, but is not practical or feasible. The quality of life of a subject that is susceptible to one or more offending allergens is greatly affected by the quality of allergy treatment management that he/she receives. It is important to monitor a subject receiving allergy treatment to determine whether and, if yes, how well the disease is kept under control, whether the subject is compliant with therapy and how well the subject responds to the chosen therapy so that the allergy/allergic disease does not exacerbate and escalates in a major, life-threatening allergic reaction/anaphylaxis. Successful therapeutic monitoring will at last not only improve the subject's quality of life, it will also reduce the subject's state, duration and frequency of morbidity and need for urgent medical intervention.

### Allergic Diseases

Allergic diseases are a group of hypersensitivity disorders that may be associated with the production of specific IgE to environmental allergens and involve IgE-mediated reactions.

Anaphylaxis is an acute, systemic hypersensitivity response to an allergen, which typically involves multiple organ systems and which, if left untreated, rapidly leads to death. Anaphylaxis can occur IgE-dependent as well as IgE-independent.

Allergic Conjunctivitis. Allergic eye disease primarily affects the conjunctiva. The signs and symptoms include itching, tearing, conjunctival edema, hyperemia, watery discharge, burning, and photophobia. Eyelid edema is also common. Symptoms are usually bilateral; however, one eye can be affected more than the other. The diagnosis of allergic conjunctivitis is usually made clinically.

Allergic rhinitis (hay fever) is one of the most prevalent allergic diseases. It generally is believed that symptoms, which include sneezing, nasal congestion and itching, and rhinorrhea primarily reflect the IgE-dependent release of mediators by effector cells in response to aeroallergens. Accordingly, symptoms may be seasonal, correlating with the presence of the offending grass, weed or tree pollens, or mold spores, or year-round in the presence of dust mites and animal dander. Typically, symptoms develop rapidly upon exposure to allergen. Nasal tissues usually exhibit marked infiltration with eosinophils and basophils.

Asthma is characterized by a predisposition to chronic inflammation of the lungs in which the airways (bronchi) are reversibly narrowed. In human allergic asthma IgE-dependent mast cell activation seems to contribute to acute allergen-induced bronchoconstriction, where the airways in the lungs are narrowed due to tightening of surrounding smooth muscles. IgE can directly or indirectly upregulate the expression of high affinity IgE receptors on basophils and mast cells, and, by binding to these receptors, prime the cells to release increased amounts of key mediators, such as histamine, IL-4 and other cytokines.

Allergic contact dermatitis, a type of eczema, is an inflammatory, chronically relapsing, non-contagious and pruritic skin disease. The skin of a patient with allergic contact dermatitis reacts overly sensitive to irritants, food, and environmental allergens and becomes red, flaky and very itchy. It also becomes vulnerable to surface infections caused by bacteria. Common allergens that can cause an allergic contact dermatitis include a) nickel (nickel sulfate hexahydrate), which is a metal alloy that is frequently encountered in jewelry and clasps or buttons on clothing; b) gold (gold sodium thiosulfate), a precious metal often found in jewelry; c) formaldehyde, which is contained as preservative in household cleaning products or paints; d) thiomerosal, a mercury preservative used in local antiseptics and in vaccines.

A food allergy is an adverse immune response to a food protein. The food protein triggering the allergic response is termed a food allergen; common food allergens are shellfish, peanuts, tree nuts, fish, milk, eggs, fresh fruits such as strawberries, mango, banana, apple. Immunoglobulin-E (IgE)-mediated food allergies are classified as type-I immediate hypersensitivity reactions. These allergic reactions have an acute onset (from seconds to one hour) and the accompanying symptoms may include angioedema (soft tissue swelling of the eyelids, face, lips, tongue, larynx and trachea); hives; itching of the mouth, throat, eyes, skin; gastrointestinal symptoms such as nausea, vomiting, diarrhea, stomach cramps, or abdominal pain; rhinorrhea or nasal congestion; wheezing, shortness of breath, or difficulty swallowing; and even anaphylaxis, a severe, whole-body allergic reaction that can result in death.

Eosinophilic esophagitis (EoE) is part of a heterogeneous group of eosinophil-associated gastrointestinal disorders that is characterized by high numbers of eosinophils infiltrating into the esophagus. While the incidence of EoE is increasing, precise mechanisms of this disease remain largely unknown, though EoE seems to be associated with allergy. Currently, esophagogastroduodenoscopy (EGD) and histological examination of esophageal biopsies are required for the diagnosis of EoE, and repeated procedures are often employed for the assessment of response to therapy. Current treatments rely on avoidance of specific food and airborne allergens in atopic patients, anti-inflammatory drugs such as glucocorticoids, or experimental drugs, such as mepolizumab. The need for less invasive procedures to diagnose and monitor EoE remains.

Auto-immune diseases are conditions in which a patient's body fails to recognize its own constituent parts as “self”, resulting in an immune response against its own cells and tissues. Many different parts of the body can be affected by auto-immune diseases, including nerves, tissues, organs, and muscles.

Anaphylaxis is defined as a serious allergic reaction that is rapid in onset and may cause death. The diagnosis of anaphylaxis is clinical and based primarily upon clinical symptoms and signs, as well as a detailed description of the acute episode, including antecedent activities and events. Anaphylaxis is a much broader syndrome than “anaphylactic shock” however, and the goal of therapy should be early recognition and treatment with epinephrine to prevent progression to life-threatening symptoms, including shock. Recognition of the variable and atypical presentations of anaphylaxis is therefore critical to providing effective therapy in the form of epinephrine, as well as reducing overreliance on less-effective medications, such as antihistamines and glucocorticoids.

Urticaria, or hives, is a common disorder affecting up to 25 percent of the population. The usual urticarial lesion is an intensely pruritic, circumscribed, raised, erythematous plaque, often with central pallor. Individual lesions may enlarge and coalesce with other lesions, and then typically will disappear over a few hours without leaving residual marks on the skin unless there is damage from scratching.

Acute allergic angioedema typically occurs within minutes to a few hours following exposure to foods, drugs, latex, or the stings of various insects. Urticaria is commonly present in this setting. It is most often seen in patients with other allergic conditions, such as atopic dermatitis, allergic rhinitis, and asthma. This type of angioedema is dependent upon the presence of IgE molecules specific to proteins in the causative agent. These specific IgE molecules bind to the patient's mast cells and trigger the reaction upon re-exposure to the antigen. Skin testing or in vitro immunoassays for specific IgE may be helpful in such cases.

### Cells of the Immune System

White blood cells (WBCs) or leukocytes are cells of the immune system that defend the human body against infectious disease and foreign materials and are often characterized as granulocytes or agranulocytes, depending on the presence or absence of granules. There are various types of leukocytes, which are all produced in the bone marrow and derived from (multipotent) hematopoietic stem cells. Leukocytes are found throughout the body, including the blood and lymphatic system.

Granulocytes feature differently staining granules in their cytoplasm when viewed under light microscopy. These granules are membrane-bound enzymes that primarily act in the digestion of endocytosed particles. There are three types of granulocytes that are named according to their staining properties: (a) neutrophils, (b) basophils, and (c) eosinophils.

Agranulocytes lack granules in their cytoplasm, but they do contain lysosomes and include lymphocytes, monocytes and macrophages.

### Granulocytes

Basophil granulocytes or basophils form part of the polymorphonuclear cell family (PMNs) together with eosinophils and neutrophils. They contain prominent cytoplasmic granules that readily stain with dyes and are therefore called basophilic (susceptible to staining by dyes) granulocytes or basophils. They are the least common of the granulocytes, representing less than 1% of the circulating white blood cells. Based on their similar morphology to mast cells, basophils have often been considered as minor and possibly redundant “circulating mast cells”. The isolation of pure basophils has been a challenge due to the low occurrence in blood and due to the fact that basophils share many physiochemical properties with other blood cells, all which considerably hampered basophil research and negatively affected interest in this type of cells.

Apart from the cytoplasmic granules, basophils constitutively express high affinity IgE receptors (Fc.epsilon.RI) and are a major source of the vasodilator histamine and other potent chemical mediators of inflammation. Like all leukocytes, basophils develop in the bone marrow, derive from hematopoietic stem cells and are released as fully mature cells with a life span of 2-3 days.

Basophils express a variety of seven membrane transverse receptors that bind chemotactic factors. Most are members of the CCR family of receptors that bind CC (cysteine-cysteine-bonded) chemokines. There are at least 27 distinct members of this subgroup reported for mammals, called CC chemokine ligands (CCL)−1 to −28. CC chemokines induce the migration of monocytes and other cell types such as natural killer (NK) and dendritic cells. Examples of CC chemokine include monocyte chemoattractant protein-1 (MCP-1 or CCL2) which induces monocytes to leave the bloodstream and enter the surrounding tissue to become tissue macrophages.

Human basophils also express a variety of cytokine receptors for interleukins, chemokines, complement, prostaglandins, and immunoglobulin FC receptors, all which help transmit the signal of cytokines in the immune system. Among these are receptors that bind to specific interleukins including interleukin-2 (IL-2), IL-3, IL-4, IL-5, and IL-33. Basophils are one of the few cells that express the IL-3 receptor, which is also known as CD123 antigen or CD 123. This characteristic has led to use CD123 expression, in addition to other CD (cluster of differentiation) markers, as a marker to specifically gate on basophils during flow cytometry analysis.

The high affinity IgE receptor (FcεR1) is thought to be the single most significant activation-linked molecule known on basophils. These receptors are comprised of four subunits: one α, one β, and two γ chains that form a tetramer structure (αβγ2). Two extracellular domains on the .alpha.-subunit allow IgE binding, whereas signaling events are initiated through immunoreceptor tyrosine-based activation motifs located within intracellular portions of the β-subunits and γ subunits. In humans, a trimeric (αγ2) form of Fc.epsilon.RI is also found on antigen-presenting cells, including Langerhans cells, monocytes and blood dendritic cells. Mast cells, eosinophils, neutrophils, platelets and dendritic cells may have these and/or functionally related receptors, too.

Basophils can infiltrate sites of many immunologic or inflammatory processes, including IgE-associated late-phase reactions and sites of chronic allergic inflammation, often in association with eosinophils. Further, basophils can be involved in IgE independent mechanisms. Generally, basophils can be activated by a number of stimuli and give rise to distinct activation pathways. Those stimuli might or might not be mediated by the high-affinity IgE receptor (FcεR1).

Basophils release several inflammatory mediators that have a role in the pathophysiology of allergic disease. The most commonly recognized inflammatory mediators are histamine and leukotriene C4 (LTC4), which cause smooth muscle contraction. It long has been thought that basophils release these substances during and/or after selectively infiltrating sites of allergic inflammation and thus contribute towards the symptoms of the late phase response. Basophils circulate in the blood under homeostatic conditions, but will migrate into tissue during the late phase response, which, upon reexposure to an offending allergen, follows the acute allergic reaction.

In humans, basophils appear to be the prime early producers of the Th2-type cytokines IL-4 and IL-13, which perform several crucial functions in initiating and maintaining allergic responses. The assumed immunomodulatory role of basophils is further supported by their ability to express CD40 ligand, which, together with IL-4 and IL-13, serve as inducers of B cell proliferation and class switching to IgE and IgG.

The cluster of differentiation (CD) system is a protocol used for the identification of cell surface molecules present on white blood cells. CD markers can act in numerous ways, often acting as receptors or ligands, by which a signal cascade is initiated, altering the behavior of a cell. Generally, a proposed surface molecule is assigned a CD number once two specific monoclonal antibodies (mAb) are shown to bind to the molecule. If the molecule has not been well-characterized, or has only one mAb, the molecule is usually given the provisional indicator “w”. The CD system nomenclature commonly used to identify cell markers thus allows cells to be defined based on what molecules are present on their surface. There are more than 350 CD molecules identified for humans, and several CD molecules are usually utilized to define a population of cells, in particular through cell sorting methods that include flow cytometry. Cell populations are usually defined using a “+” or “−” symbol to indicate whether a certain cell fraction expresses or lacks a certain CD molecule. For example, all hematopoietic cells express CD45, and thus are defined as CD45+. Furthermore, all granulocyte cells express in addition CD15, so they are defined as CD45+, CD15+.

Most CD molecules have important functions beyond their use as cell surface markers; for example CD 123 that is expressed by basophils, as mentioned supra, induces tyrosine phosphorylation with the cell and promotes proliferation and differentiation within the hematopoietic cell lines. CD203c is another CD marker that is expressed on the cell surface and within intracellular compartments of basophils, mast cells and precursors of these cells. CD203c detection by flow cytometry has been used to specifically identify basophils within a mixed leukocyte suspension, since its expression is unique to basophils among the cells circulating in blood. The expression of CD203c is both rapidly and markedly upregulated following IgE-dependent activation. CD63, a cell surface glycoprotein of the transmembrane 4 superfamily, is also upregulated following IgE-dependent cell activation, however, like CD203c, is not specific enough to serve reliably as a diagnostic marker for the diagnosis of IgE-mediated allergic reactions. Apart from their marker function, CD molecules have other tasks that include the facilitation of cell attachment, phagocytosis and chemotaxis as well as the recruitment of kinases.

Eosinophil granulocytes or eosinophils are primarily tissue-dwelling granulocytes that are recruited to sites of acute inflammation, and are seen most prominently in response to respiratory, gastrointestinal, and dermatologic allergens, as well as to generalized infection with helminthic parasites. Eosinophils have been found to have innate capacities to secrete differentially multiple preformed cytokines. Eosinophil-associated allergic inflammatory diseases notably occur in the airways and include asthma and rhinorrhea. Eosinophils that are recruited into the mucosal airway tissues and secretions are positioned to encounter aeroallergens where they may function as antigen-presenting cells. For example, in humans, blood eosinophils, which normally do not display Major Histocompatibility Complex (MHC) class II proteins, can be induced to do so by stimulation with cytokines, including GM-CSF, IL-3, 11-4, 11-5 and interferon-.gamma. (IFN-.gamma.). On most immune system cells, specifically on antigen-presenting cells, MHC class II proteins contain .alpha. and .beta. chains and present antigen fragments to T-helper cells by binding to the CD4 receptor on the T-helper cells. Moreover, human eosinophils recruited into the airways, as evidenced in the sputum of asthmatics and in lung lavages after allergen challenges, typically express MHC II proteins. Unlike the gastrointestinal tract, where eosinophils normally are found and might be exposed to gut-derived antigens, eosinophils are not abundant in the normal lungs or airways. In contrast, recruitment of eosinophils into the upper and lower airways is a frequent concomitant of allergic inflammation. It is in this setting of allergic airways diseases that recruited eosinophils might function not simply as effectors of local inflammation, but also as “inflammatory” full-function antigen-presenting cells in processing and presenting airway antigens. In the context of allergic upper and lower airways diseases in which eosinophils are characteristically elicited, the capacity of eosinophils to serve as additionally recruited “inflammatory” full-function antigen-presenting cells could be pertinent to antigen-elicited immune responses in the airways of those with often chronic, eosinophilic allergic diseases.

Neutrophil granulocytes or neutrophils are the most abundant type of white blood cells in mammals by representing between 40% and 50% of the circulating leukocyte population and form an essential part of the innate immune system. The name, neutrophil, derives from particular staining characteristics on histological and/or cytological preparations. Whereas basophils stain dark blue and eosinophils stain bright red, neutrophils stain a neutral pink. Neutrophils are normally found in the blood stream. However, during the acute phase of inflammation, neutrophils are one of first-responders of inflammatory cells to migrate toward the site of inflammation, first through the blood vessels, then through interstitial tissue, following chemical signals such as IL-8 and IFN-gamma in a process called chemotaxis. Neutrophils are recruited to the site of injury within minutes following trauma and are the hallmark of acute inflammation.

Neutrophils are crucial to both immunity and inflammation, and prolonged neutropenia (a decrease in the number of neutrophils) leads inevitably to life-threatening situations as a result of insufficient protection against infections. Circulating neutrophils are quiescent cells with only the potential to mediate a wide range of inflammatory activities; this potential is realized when neutrophils are activated by agents including, but not limited to, leukotriene B4 (LTB4), complement fragment C5a, platelet activating factor (PAF), histamine, IFN-.gamma., granulocyte colony-stimulating factor (G-CSF), granulocyte-macrophage colony-stimulating factor (GM-CSF), IL-8, tumor necrosis factor-.alpha. (TNF-.alpha.) and different chemoattractants. Those activating agents transmit signals to neutrophils via interaction with specific cell surface receptors, many of which interact with intracellular G proteins. G proteins catalyze the hydrolysis of guanosine triphosphate (GTP) to guanosine diphosphate (GDP) and inorganic phosphate, and initiate a series of events including activation of phospholipase C, initiation of calcium fluxes and membrane depolarization. Once activated, neutrophils are able to adhere to endothelial cells, migrate through the endothelial barrier, and ingest and attempt to destroy pathogens, foreign bodies, and remnants of tissue damage. Activated neutrophils exhibit an enhanced response to subsequent stimuli.

Agranulocytes are characterized by the absence of stainable granules in their cytoplasm, but they do lysosomes. Agranulocytes include lymphocytes, monocytes and macrophages.

### Flow Cytometry

The availability of monoclonal antibodies directed against cell surface markers of interest made flow cytometry an ideal approach for studying activation of granulocytes.

Flow cytometry is a technique for counting and examining small particles such as cells by suspending them in a stream of fluid and passing them by an electronic detection apparatus. It allows simultaneous multiparametric analysis of the physical and/or chemical characteristics of each individual particle or cell. Briefly, a beam of light (usually laser light) of a single wavelength is directed onto a hydrodynamically-focused stream of fluid. A number of detectors are aimed at the point where the stream passes through the light beam: one in line with the light beam (forward scatter), several in perpendicular position (side scatter) and at least one fluorescence detector. Each suspended cell (from 0.15 μm-150 μm) passing through the light beam scatters the light in some way, and fluorescent molecules (naturally occurring or as part of an attached label or dye) may be excited into emitting light at a longer wavelength than the light source. This combination of scattered and fluorescent light is recorded by detectors. The forward scatter correlates with the cell volume, while the side scatter depends upon the inner complexity of the cell (such as shape of the nucleus). The data generated by flow-cytometers can be plotted in a single dimension to produce a histogram or in two-dimensional or three dimensions plots. The regions on these plots can be sequentially separated, based on fluorescenc intensity, by creating a series of subset extractions, termed “gates.” Specific gating protocols exist for diagnostic and clinical purposes, especially for hematology. There are also flow cytometers who only use light scatter, without fluorescence, for the analysis.

Fluorescence activated cell sorting (FACS) is a specialized type of flow cytometry and provides a method of sorting a heterogeneous mixture of cells into two or more containers, a single cell at a time, based upon the specific light scattering and fluorescent characteristics of each cell. The use of multicolor, multiparameter FACS requires primary conjugated antibodies at defined fluorophore-to-protein (FTP) ratios.

The cell suspension is entrained in the center of a narrow, rapidly flowing stream of liquid and the flow is arranged so that there is a large separation between cells relative to their diameter. The stream of individual cells passes through a fluorescence detector, and an electrical charge is assigned to each cell (based on the cell's fluorescence) just at the point where the stream breaks into individual droplets (usually via a vibrating mechanism) such that there is a low probability that more than one cell per droplet occurs. Each charged droplet (containing an individual cell) may be sorted, via electrostatic deflection, into separate containers.

The surfaces of all cells in the body are coated with specialized protein receptors that selectively can bind or adhere to other signaling molecules. These receptors and the molecules that bind to them are used for communicating with other cells and for carrying out proper cell functions in the body. Each cell type has a certain combination of receptors or cell markers on its surface that makes it distinguishable from other kinds of cells. Cells may, for example, be fluorescently or radioactively labeled. The most commonly used labeled molecules are antibodies; their specificity towards certain surface markers on a cell surface allows for more precise detection and monitoring of particular cells. The fluorescence label that can be used will depend upon the lamp or laser used to excite the fluorochromes and on the detectors available.

The development of flow-cytometry based approaches for the identification of activation markers, via measurement of enzymatic and surface marker profiles, has allowed for accelerated association of surface topologies with disease states. Studies that involve the triggering of cells to respond to environmental stimuli, such as an allergen or drug action, and the activation phenotypes associated with such agitation, allow for clearer resolution of the underlying activation states and provide for more distinct classification of allergic disease outcomes. Allergy is a dynamic event, and as such, static views of basal states would be considered insufficient for determination of an activated state, therefore rendering correlations to clinical outcomes less meaningful. Fractionation of cell populations with flow cytometry is well suited to address activation markers and intracellular markers in the context of allergic disease, because it can simultaneously discern multiple surface markers within complex cellular populations.

### Utility

The analysis of subpopulations of white blood cells (leukocytes) in blood or bone marrow is of particular interest for the evaluation of immune system disorders and immune system-related diseases, especially allergic diseases. Basophils, neutrophils and eosinophils play important roles in the allergic response to an offending allergen and/or environmental stimulus. Upon activation by an allergen and/or stimulus, basophils, eosinophils and/or neutrophils can exhibit changes on their cell surfaces and/or inside the cell which can be detected, classified and correlated with the particular allergen or stimulus with the objective to identify an offending allergen or stimulus to which a mammalian subject is allergic.

### Blood-Based Allergen Testing

Carrying out testing for offending allergens in blood cells offers the great advantage that a blood sample can quickly and without much discomfort be obtained from a mammalian subject. Furthermore, the testing for an offending allergen is done by ex vivo activation, which means that the offending allergen is not directly ingested (or otherwise administered) by the mammalian subject, but the offending allergen is added to an isolated fraction of a blood sample drawn from the particular mammalian subject. This way, the subject is in no way endangered to experience a potentially life-threatening allergic reaction, as it would be if the subject had to ingest an offending allergen, as is the case in in-vivo food challenge tests, or if the subject had to be externally administered an offending allergen, as is the case in conventional allergy skin tests.

An in-vivo food challenge test is generally carried out in a double-blind, placebo-controlled fashion to determine the offending allergen. This test is not only difficult to administer, but it is also very time-consuming and, most importantly, potentially highly dangerous since it can result in anaphylactic shock and even death, if treatment is not initiated quickly.

Conventional allergy skin tests, where several potentially offending allergens are subcutaneously administered together with histamine as positive control to a subject, are less dangerous in that they usually don't evoke a life-threatening anaphylactic shock situation. However, the subcutaneous administration is usually discomforting, in particular for a pediatric subject, and often inconclusive, since the extent of a potentially positive indication on the skin (‘skin reaction’), as determined when the spot where a potential allergen or stimulus was administered start to inflame or appears inflamed within a certain time (usually 15-60 minutes), does not necessarily correlate with a true allergic response. Hence, a weak skin reaction can still be followed by a strong allergic response, whenever the subject is exposed to the offending allergen in a real life situation. Vice versa, a strong skin reaction that indicates a strong allergy to the offending allergen, can nevertheless be followed by only a slight, subtle, possibly not even noticeable allergic response, whenever the subject is exposed to the offending allergen or stimulus in a real life situation. Rapid, Safe and Reliable Blood-Based Susceptibility Testing to Offending Allergens

Other approaches that attempt to monitor or determine the activation status of granulocytes are often based on simply counting white blood cells upon granule-staining by manual or automatic means or separating white blood cells by density gradient and subsequent cell sorting. While these methods are time-consuming and can only provide an estimate, they are seriously deficient in their reliability because those methods don't distinguish well live from dead cells and don't provide any insights into what is going on inside the cells, in particular, inside of granulocytes.

The described invention, in contrast, provides the ability to monitor cell activation in biological samples such as whole blood, in particular in white blood cells such as live basophils and live eosinophils that are specifically gated and labeled for the determination of their activation status. The described invention, furthermore, allows for a safe and time-efficient evaluation without endangering the subject who is undergoing the testing, by measuring said activation status ex vivo and in a straight-forward manner from blood sampling to cell separation, ex-vivo activation and determination of activation status. By specifically evaluating cell surface markers in activated granulocytes in comparison to non-activated granulocytes from the same whole blood sample that was provided by the subject who is undergoing the testing, the monitoring and evaluation of response of the subject to an offending allergen or stimulus is reliable and sensitive.

### Monitoring of Allergy Treatment Success

Consistent allergen avoidance would be ideal and would obviate the need for allergy treatment, but is not practical or feasible. The quality of life of a subject that is susceptible to one or more offending allergens is greatly affected by the quality of allergy treatment management that he/she receives. It is important to monitor a subject receiving allergy treatment to determine whether and, if yes, how well the disease is kept under control, whether the subject is compliant with therapy and how well the subject responds to the chosen therapy so that the allergy/allergic disease does not exacerbate and escalates in a major, life-threatening allergic reaction/anaphylaxis. Successful therapeutic monitoring will at last not only improve the subject's quality of life, it will also reduce the subject's state, duration and frequency of morbidity and need for urgent medical intervention. Certain embodiments of the present invention describe the ex-vivo detection of basophil activation of a subject, that is under ongoing allergy treatment, at baseline level, i.e. without any external stimulation of an offending allergen in order to determine and monitor that subject's responsiveness to the allergy treatment. The monitoring requires repeated testing at specified time interval (daily, weekly, biweekly, monthly and so forth) and comparison of the test results to enable a reliable determination of therapy progress and success

Diseases and disorders where this method will be useful are food allergy, airborne allergy, drug-induced allergy (suspected, based on medical history, or more importantly, in absence of a known predisposition), anaphylaxis, asthma and other immune disorders.

Particular embodiments of the present invention use very small volumes of whole blood (100 μl or less per assay) and so are also suitable for studies in infants, children, healthy and sick individuals. The blood samples are generally obtained by venipuncture and are immediately put on ice and further processed at 4° C. to preserve optimal cellular viability and functionality.

The above-described methods may be performed on a mammalian subject, e.g., a human being or some other member of a species of mammalian origin, who is: a) suspected of having an allergy to some offending allergen or stimulus, based on medical history or known predisposition or b) is not suspected of having an allergy to some offending allergen or stimulus, in the absence of a known predisposition, to determine if that subject has an allergy to some offending allergen or stimulus.

Since the methods of the present invention to evaluate a subject for any kind of allergic response to an offending allergen or stimulus only require one drop (100 μl or less) of blood per analysis, they are ideally suited for testing all type of subjects (e.g. children, small children, infants as well as sick subjects who cannot afford to provide much blood for analysis).

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. In the following, examples and experimental procedures will be described to illustrate parts of the invention.

### EXAMPLES

The following examples are put forth so as to provide those of ordinary skill in the art with a complete disclosure and description of how to make and use the present invention; they are not intended to limit the scope of what the inventors regard as their invention. Unless indicated otherwise, part are parts by weight, molecular weight is average molecular weight, temperature is in degrees Centigrade, and pressure is at or near atmospheric.

### Example 1

When granulocytes are incubated with an offending allergen, the CD203c up-regulation on the basophil cell surface provides a marker for the IgE mediated human allergies specific to food, environment, microbial, nano-particles, metals and drug related antigens. Patients who have been previously diagnosed with the peanut allergies by skin prick tests were examined for their CD203c inducible expression at the time of in vitro activation with the offending allergen. Whole blood (5 ml) was first spun at 300×g (0 ac/dc brakes). Cells were collected with the plasma, and mixed in a separate tube. 100 μl of the cells were transferred to a single well of a 96 well plate that was pre-incubated with offending allergen (3 μl) extracts and control samples. Samples were incubated for 20 minutes.

Cells were stained for markers of interest and analyzed by flow cytometry. Basophil characterization was done by gating on CCR3 high and SSC low cells. Eosinophils were excluded on the basis of high granularity, and T cells were excluded on the basis of low granularity and low CCR3 expression. CD203c expression was analyzed by staining with anti-CD 203c antibodies and flow cytometry, comparing cells before and after stimulation.

A food challenge was performed for 25 subjects with food allergies at baseline, and the basophil activation test (CD203c and CD63 respectively) results were compared to the grade of the clinical reaction during the food challenge.

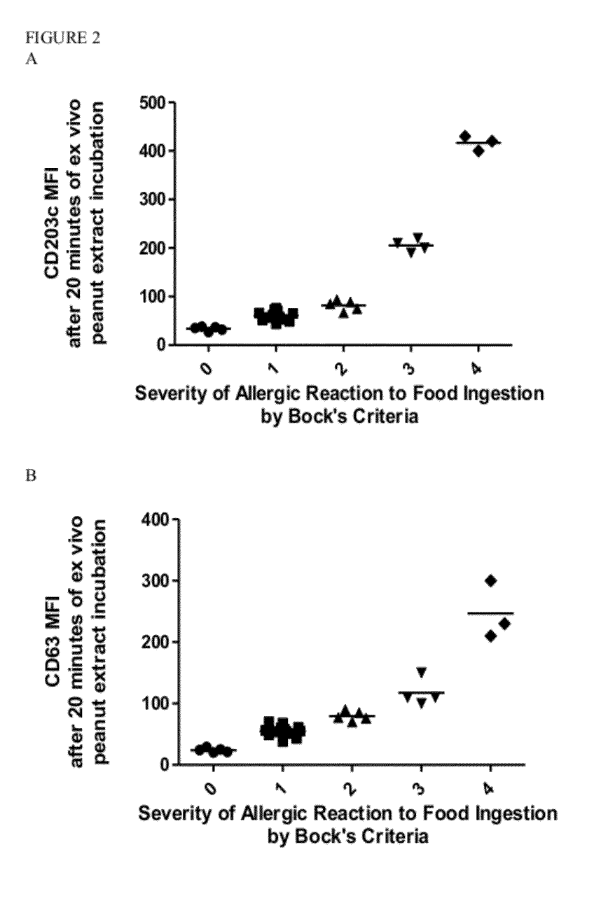

As shown in FIG. 2, there was a strong positive correlation between the severity of the subject's allergic reaction to a food challenge with peanut (as measured by modified Bock's criteria) and the levels of expression of CD203c or CD63 on the surface of the subject's blood basophils after 20 min of ex vivo stimulation with food allergen extract.

### Example 2

### Small Volume Analysis

The basophil activation based method can be used in the diagnosis and monitoring of allergies or related diseases in humans. When blood cells are incubated with allergens, basophils can be specifically activated and express various cell surface markers such as CD203c and CD63. Induction of these cell surface molecules on basophils indicates if the subject is allergic to any particular antigen. This protocol can be used for human allergies specific to food, environment, microbial, nano-particles, metals and drug related antigens.

While this protocol is written for diagnosis and monitoring of allergic diseases, it can also be used for translational studies to investigate functions of granulocytes. Parameters include number of cells, antibody concentration and basophils activation.

Materials

### Azide Buffer: 0.05% NaN₃, 1% BSA in Sterile PBS

### Anti-Human CD203c Antibody-APC Conjugated (Cat No. 324609, Biolegend, USA)

### Anti-Human CD63 Antibody-FITC Conjugated (Cat No. 557288, BD Pharmingen, USA)

### Anti-Human CCR3 Antibody-PE Conjugated (Cat No. 310706, Biolegend, USA)

Allergens Extracts—Purchased from GREER® Labs
Negative control for Allergens-50% Glycerine/50% Cocas from GREER® Labs

### Anticoagulant-2 mM EDTA or Heparin

### RBC Lysis Buffer: Ammonium Chloride Solution (Cat No. 07800, STEMCELL)

96 Well U bottom plate (Cat No. 353227, BD Falcon)

Instruments

Centrifuge: Allergra X-22R centrifuge, Beckman Coulter
Lancing Device Accu-Check Multiclicx lancing device kit (Cat No. 04466152160, Roche)

### BD Facscalibur Flow Cytometer (BD Biosciecnes)

### BD LSR II (BD Biosciecnes)

Experimental Durations

### Whole Blood Activation: 10 to 20 Minutes

### Antibody Staining: 20 Minutes

### RBC Lysis: 15 to 20 Minutes

Skin Prick Test. Add 3 μl of allergen extract (GREER® Labs) and anticoagulant (10 μl of Heparin (3 IU/ml) or 10 μl of 20 mM EDTA) in each well of the cell culture treated 96 well plate. Control allergens are similarly added. By using the accu-Check Multiclicx lancing device, 1-2 drops of blood is collected directly in to the each well, containing the allergen and the anticoagulant. Mix with the pipette gently and incubate for 10 to 20 minutes in 37° C., 5% CO₂ humidified incubator.

Add 25 μl of diluted CCR3-PE (10 μl/well of 25 μg/ml), CD63-FITC (10 μl/well of 25 μg/ml) and CD203c-APC (5 μl/well of 25 μg/ml). Any dilutions required should be made in 0.05% NaN₃, 1% BSA in PBS. Incubate on ice for 20 minutes in dark. Basophils are recognized as CCR3^{h} μg^{h}/SSC″ cells.

Alternatively, add 25 μl of diluted HLA-DR-FITC (10 μl/well of 25 μg/ml), CD123-PerCP5.5 (10 μl/well of 25 μg/ml), CD63-PE (10 μl/well of 25 μg/ml) and CD203c-APC (5 μl/well of 25 μg/ml). Any dilutions required should be made in 0.05% NaN₃, 1% BSA in PBS. Incubate on ice for 20 minutes in dark. Basophils are recognized as HLA-DR^{negative}/cD123^{positive} cells.

Spin the plates at 2000 rpm for 5 minutes and carefully collected the plasma for further analyses. Add 200 μl of Ammonium Chloride buffer, resuspend the cells, and incubate at ice for 20 minutes. Spin the plates at 2000 rpm for 5 minutes and washed the cells 2 times with 0.05% NaN₃, 1% BSA in PBS buffer. Resuspend the cells in 200 μl of PBS for flow cytometry analysis either by BD LSR11 or BD Facscalibur. Flow cytometry results are analyzed by Flow Jo Software (Tree Star, Inc) or FCS Express Software (De Novo Software).

In an alternative method, the samples are frozen for later analysis. In this method, the activation step with allergen is performed as described above, but the cells are then washed with 100 μl 0.05% NaN₃, 1% BSA in PBS Buffer or 2 mM EDTA in PBS buffer to stop the activation reaction and keep on ice. Add 100 μl of 10% DMSO in FBS, resuspend the cells and transfer to the cryotubes and store at −80° C. On the day of analyses, thaw the cells by placing cryovial in 37° C. Optionally, to lyse RBC, when only a small piece of ice remains, add 100 μl of RPMI and 10% FCS dropwise. Spin the tube at 2000 rpm for 5 minutes and discard the supernatant. Perform the staining steps to complete the analysis.

### Example 3

### Larger Volume Analysis

Materials

Blood collection tubes: BD Vacutainer Sodium Heparin tube or BD Vacutainer K₂ EDTA tube

### Azide Buffer: 0.05% NaN₃, 1% BSA in Sterile PBS

### Anti-Human CD203c Antibody-APC Conjugated (Cat No. 324609, Biolegend, USA)

### Anti-Human CD63 Antibody-FITC Conjugated (Cat No. 557288, BD Pharmingen, USA)

### Anti-Human CCR3Antibody-PE Conjugated (Cat No. 310706, Biolegend, USA)

Allergens Extracts—Purchased from GREER® Labs Negative control for Allergens-50% Glycerine/50% Cocas from GREER® Labs

### Anticoagulant-2 mM EDTA or Heparin

96 Well U bottom plate (Cat No. 353227, BD Falcon)

Instruments

Centrifuge: Allergra X-22R centrifuge, Beckman Coulter

### BD Facscalibur Flow Cytometer (BD Biosciecnes)

### BD LSR II (BD Biosciecnes)

Experimental Durations

### Whole Blood Activation: 10 to 20 Minutes

### Antibody Staining: 20 Minutes

Method. About five ml of blood is collected in heparin or EDTA coated tubes. (100 μl of the whole blood can be taken at this step and used in other DAT protocol similar to skin prick test protocol). Spin the blood containing tubes at 300 g for 6 minutes without ac/dc brakes or 0 brakes by using the Allergra X-22R centrifuge. Collect the plasma containing cells (top layers) and mix in the separate tube. Add the 100 μl of the cells to the single well of a 96 well cell culture treated plate (BD Falcon 353227), pre-incubated with 3 μl of allergen (GREER® Labs) or controls (GREER® Labs) and incubated for 20 minutes in 37° C., 5% CO₂ humidified incubator. Centrifuge the plates at 2000 rpm for 5 minutes and discard or collect the plasma for further analyses.

Add 50 μl of diluted CCR3PE (10 μl/well of 25 μg/ml), CD63-FITC (10 μl/well of 25 μg/ml) and CD203c-APC (5 μl/well of 25 μg/ml). Any dilutions required should be made in 0.05% NaN₃, 1% BSA in PBS. Incubate on ice for 20 minutes in dark. Basophils are recognized as CCR3^{high}/SSC^{low} cells.

Alternatively, add 50 μl of diluted HLA-DR-FITC (10 μl/well of 25 μg/ml), CD123-PerCP5.5 (10 μl/well of 25 μg/ml), CD63-PE (10 μl/well of 25 μ/ml) and CD203c-APC (5 μl/well of 25 μg/ml). Any dilutions required should be made in 0.05% NaN₃, 1% BSA in PBS. Incubate on ice for 20 minutes in dark. Basophils are recognized as HLA-DR^{negative}/CD123^{positive} cells.

Spin the plate at 2000 rpm for 5 minutes and wash two times with 0.05% NaN₃, 1% BSA in PBS buffer. Resuspend the cells in 200 μl of PBS for flow cytometry analysis either by BD LSR11 or BD Facscalibur. Flow cytometry results are analyzed by Flow Jo Software (Tree Star, Inc) or FCS Express Software (De Novo Software).

If it is desirable to freeze the sample for later analysis, they can be treated as described in Example 2.

### Example 4

### Whole Blood Assay

Add 100 to 200 μl whole blood to wells (or FACS tubes), using a minimum of 50 μl. Add allergens (3 uL food or 1 uL IgE, or 3 uL PBS), resuspend. Incubate for 20 minutes in the incubator. Make up antibody cocktail (10 uL per antibody×4 Antibodies=40 uL per sample). Remove samples from incubator, add 1 mL cold RoboSep (with 1 mM EDTA) to sample. Spin samples at 1500 rpm for 5 minutes. Aspirate supernatant, resuspend in antibody cocktail (40 uL). Incubate 20 minutes on ice in the dark. Move samples to FACS tubes and add 1-2 mL ammonium chloride. On ice, flick the tube and incubate for 5-10 min. Sample should turn transparent. Add 2 mL of Staining/Wash Buffer and spin 1500 rpm for 5 minutes. Discard supernatant, wash again in 2 mL of staining/wash buffer. Spin 1500 rpm for 5 minutes, discard supernatant. Go to FACS Calibur.

If analyzing on a plate: Spin 1500 rpm, 5 minutes, discard supernatant with pipette tip. Add 200 uL ammonium chloride for 20 minutes on ice. Spin 5 minutes 1500 rpm, discard supernatant. Resuspend in azide buffer, spin 1500 rpm 5 minutes, discard supernatant. Add 200 uL azid buffer, transfer to FACS tubes for Calibur analysis.

Antibody panel:

| Basophils: | Eosinophils: |
|---|---|
| CD63-FITC | CD16-FITC |
| HLADR-PE | CD3-PE (neg) |
| CD123-PerCpCy5.5 | CD66b-PerCpCy5.5 |
| | (positive) |
| CD203c-APC | CCR3-APC |

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope.

What is claimed is:

1. A method for determining a subject's susceptibility to an allergic reaction against an allergen of interest, the method comprising:
collecting a blood sample from the subject;
contacting the blood sample in vitro with the allergen of interest; or a positive control antigen;
determining expression levels of a basophil cell surface activation marker;
comparing the expression levels to a background level of expression of the marker in the absence of the allergen or positive control;
wherein increased expression of the marker in the presence of the allergen of interest is indicative that the subject has an allergic response to the allergen of interest.

2. The method of claim 1, wherein the marker is one or more of CD203c, CD63, CD13, CD107a, CD164, CD80, CD86, CD40L, HLA-DR, CD123, CRTH2, Ph-CREB, Ph-STAT5, Ph-S6rp, Ph-eIF4E, CREB, mTOR pathway proteins, and phosphorylation related markers.

3. The method of claim 1, wherein the marker is one or both of CD203c and CD63.

4. The method of claim 1, wherein determining the level of expression of a basophil cell surface activation marker comprises contacting the whole blood sample with an antibody specific for said basophil cell surface activation marker; and quantitating the presence of antibody bound to basophils.

5. The method of claim 4, further comprising contacting the whole blood sample with an antibody that distinguishes basophils from other blood cells.

6. The method of claim 5, wherein the antibody that distinguishes basophils from other blood cells is CCR3.

7. The method of claim 4, wherein the quantitating is performed by flow cytometry.

8. The method of claim 7, wherein the analysis is gated on basophils.

9. The method according to claim 1, wherein the allergen of interest is a food-based allergen.

10. The method according to claim 1, wherein the allergen of interest is an airborne allergen or environmental allergen.

11. The method according to claim 1, wherein the allergen of interest is a nano-particle.

12. The method according to claim 1, wherein the allergen of interest is a metal or metal alloy.

13. The method according to claim 1, where in the allergen of interest is a drug.

14. The method according to claim 1, where in the allergen of interest is an animal allergen.

15. The method according to claim 1, where in the allergen of interest is an insect allergen.

16. The method according to claim 1, wherein the allergen is related to humidifiers and air conditioners.

17. The method according to claim 1, wherein the subject is evaluated for development of tolerance as a result of immunotherapy or related allergic treatment.

18. The method according to claim 1, wherein the positive control antigen is anti-IgE, anti-IgG, anti-IgD antibodies, fMLP, cytokines, IL-3, IL-18, IL-33, histamine, anti-Fc receptor antibodies, PMA/Ionomycin, PMA/ca1, proteases enzymes, papain, TLR receptor/ligand or antibodies against TLR or agonists, complement factors, antigens from helmiths, ROS pathway markers, or other intracellular or extracellular markers that are involved in the basophil activation or degranulation.

19. The method of claim 1, wherein an enhancing agent is added to the whole blood sample.

20. The method according to claim 15 wherein the enhancing agent is IL-3.
